(19) United States
(12) Patent Application Publication
(10) Pub. No.: US 2018/0025514 A1
(43) Pub. Date: Jan. 25, 2018

(54) TOMOGRAPHY SYSTEM

(71) Applicants: Purdue Research Foundation, West Lafayette, IN (US); HIGH PERFORMANCE IMAGING, INC., West Lafayette, IN (US)

(72) Inventors: Charles Addison Bouman, West Lafayette, IN (US); Samuel Pratt Midkiff, West Lafayette, IN (US); Sherman Jordan Kisner, West Lafayette, IN (US); Xiao Wang, West Lafayette, IN (US)

(73) Assignees: Purdue Research Foundation, West Lafayette, IN (US); HIGH PERFORMANCE IMAGING, INC., West Lafayette, IN (US)

(21) Appl. No.: 15/550,980

(22) PCT Filed: Feb. 16, 2016
(86) PCT No.: PCT/US16/18123

§ 371 Date: Aug. 14, 2017

Related U.S. Application Data
(60) Provisional application No. 62/247,771, filed on Oct. 29, 2015.
(60) Provisional application No. 62/116,235, filed on Feb. 13, 2015.

(51) Int. Cl. G06T 11/00; G06T 15/08; A61B 6/00; G06T 1/60

(52) U.S. Cl. CPC G06T 11/008; G06T 1/60; G06T 11/006; G06T 15/08; A61B 6/5205; G06T 2211/424; G06T 2211/421; A61B 6/032

(57) ABSTRACT

Systems and methods for MBIR reconstruction utilizing a super-voxel approach are provided. A super-voxel algorithm is an optimization algorithm that, as with ICD, produces rapid and geometrically agnostic convergence to the MBIR reconstruction by processing super-voxels which comprise a plurality of voxels whose corresponding memory entries substantially overlap. The voxels in the super-voxel may also be localized or adjacent to one another in the image. In addition, the super-voxel algorithm straightens the memory in the “sinogram” that contains the measured CT data so that both data and intermediate results of the computation can be efficiently accessed from high-speed memory and cache on a computer, GPU, or other high-performance computing hardware. Therefore, each iteration of the super-voxel algorithm runs much faster by more efficiently using the computing hardware.

### CROSS REFERENCE TO RELATED APPLICATIONS

The present patent application is related to and claims the priority benefit of U.S. Provisional Patent Application Ser. No. 62/116,235, filed Feb. 13, 2015 and U.S. Provisional Patent Application Ser. No. 62/247,771, filed Oct. 29, 2015. The contents of both of these applications are hereby incorporated by reference in their entirety into the present disclosure.

### TECHNICAL FIELD

The present application relates to tomography imaging devices, e.g., computed tomography devices, and to tomography control systems.

### BACKGROUND

Computed tomography (CT) is a general method for non-destructive imaging of objects or organisms using X-ray transmission measurements. CT scanners are used in a wide array of applications including healthcare, industrial inspection, transportation security, electron microscopy, and synchrotron imaging. In each case, a major challenge is to compute an accurate reconstruction of an object cross section from the available measurements. Reconstructed image quality may be limited due to many causes including limited amounts of data, limited data quality, excessive sensor noise, limited sensor resolution, motion blur, incomplete projections, missing projections, detector cross-talk, X-ray source spot size, X-ray source spectrum, approximations in material models, beam hardening, and X-ray scatter.

Model-Based Iterative Reconstruction (MBIR) can be used to improve the quality of reconstructed images in applications using X-ray computed tomography. However, MBIR is known to be very computationally expensive. The MBIR reconstruction is computed by minimizing a cost function, and a number of methods exist for performing this minimization. Some reconstruction methods, such as iterative coordinate descent (ICD), have very rapid convergence and are agnostic to the geometry of the CT scanner, which makes ICD desirable for use in applications such as security CT scanners. However, a disadvantage of ICD is that it tends to require memory access patterns that do not align with the organization of typical computer system caches. In practice, this poor alignment of memory accesses can dramatically slow down the rate at which data is accessed, and therefore can severely reduce the speed at which the ICD algorithm can run on high performance computers and graphical processing units (GPUs). Therefore, improvements are needed in the field.

### SUMMARY

According to one aspect, a tomography system is disclosed, comprising one or more computer processors having a first memory, the first memory having a first access speed, a second memory having a second access speed slower than the first access speed, and one or more program modules stored on a third memory and executable by the one or more processors to perform operations. The performed operations may comprise receiving measured data from a tomography scanner, storing the measured data in the second memory, and creating an initial image from the received measured data, the initial image comprising a plurality of voxels, and updating a super voxel in the initial image. The super voxel may comprise a plurality of voxels whose corresponding data locations in the second memory substantially overlap, with the updating performed by retrieving the corresponding data from the second memory and storing said corresponding data in the first memory, the corresponding data comprising a subset of the measured data. The first memory may be organized in cache lines, and the updating may include rearranging the portions of the measured data so that successive accesses to the measured data in the first memory by the at least one of the processors proceeds sequentially along each of the cache lines. The third memory may be separate from the first memory and second memory or may be part of the first memory or second memory. The voxels of the super voxel may also be substantially adjacent in the image. The measured data may form a sinogram. The performed operations may also include storing the measured data in a memory buffer, the memory buffer located in the first memory or the second memory, the measured data corresponding to the super voxel.

According to another aspect, a method for iterative reconstruction of computer tomography data is disclosed, comprising receiving computer tomography measured data, creating an initial image from the measured data using a computer processor, and updating spatially localized super-voxels in 2 or more dimensions, each super-voxel corresponding to a plurality of voxels whose corresponding measured data in memory substantially overlap.

According to another aspect, a method for iterative image reconstruction of computer tomography data is disclosed, comprising receiving computer tomography measured data and creating super-voxels with a size and shape so that memory access time is reduced, wherein the super-voxels corresponding measured data are localized in memory.

According to another aspect, a method for forward or back projection of computer tomography data is disclosed, comprising receiving computer tomography measured data and reorganizing stored measured data corresponding to a super-voxel so that the associated stored measured data are straightened to be in memory locations to allow efficient access by the computer, the super voxel comprising a plurality of image voxels whose corresponding stored measured data substantially overlap.

### BRIEF DESCRIPTION OF THE DRAWINGS

In the following description and drawings, identical reference numerals have been used, where possible, to designate identical features that are common to the drawings.

FIG. 1 is a diagram showing components and operation of a first-generation computed tomography (CT) scanner useful with various aspects.

FIG. 2 is a diagram showing components and operation of a third-generation CT scanner useful with various aspects.

FIG. 3 is a system block diagram according to various aspects.

FIG. 4 shows an example projection of a single voxel from image space to sinogram space.

FIG. 5 shows an example memory organization according to prior schemes.

FIG. 6 shows an example memory organization and use according to various aspects.

FIG. 7 shows an example memory organization and use according to various aspects.

FIG. 8 shows an example of memory organization and use according to various aspects.

FIG. 9 is a diagram showing components of an example CT data-processing system according to various aspects.

FIG. 10 is a diagram showing components of an example CT data-processing system according to various aspects.

FIG. 11 shows an example memory organization utilizing Z-slice parallelization according to various aspects.

FIG. 12 shows an example super-voxel layout according to various aspects.

FIG. 13 shows an example augmented super-voxel buffer using inter-SV parallelism.

FIG. 14 shows a super voxel buffer corresponding to a particular super-voxel according to various aspects.

FIG. 15 shows transposition of individual portions of a super voxel buffer according to various aspects.

The attached drawings are for purposes of illustration and are not necessarily to scale.

### DETAILED DESCRIPTION

X-ray computed tomography, positron-emission tomography, and other tomography imaging systems are referred to herein generically as “CT” systems.

Various aspects relate to Super-Voxel Buffering for Fast Inversion. The terms “I,” “we,” “our” and the like throughout this description do not refer to any specific individual or group of individuals.

Throughout this description, some aspects are described in terms that would ordinarily be implemented as software programs. Those skilled in the art will readily recognize that the equivalent of such software can also be constructed in hardware, firmware, or micro-code. Because data-manipulation algorithms and systems are well known, the present description is directed in particular to algorithms and systems forming part of, or cooperating more directly with, systems and methods described herein. Other aspects of such algorithms and systems, and hardware or software for producing and otherwise processing signals or data involved therewith, not specifically shown or described herein, are selected from such systems, algorithms, components, and elements known in the art. Given the systems and methods as described herein, software not specifically shown, suggested, or described herein that is useful for implementation of any aspect is conventional and within the ordinary skill in such arts.

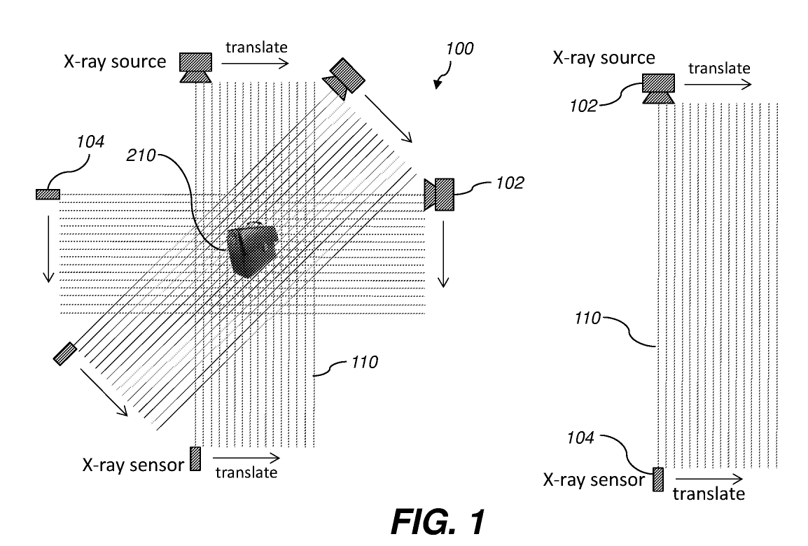

FIG. 1 shows a diagram of a first generation CT scanner 100 according to one embodiment, which includes an X-ray source 102 and a single detector 104, which are mounted on a rotating gantry and rotated about an object 210. The X-ray source 102 and detector 104 are set at a series of angles, and at each angle, the source 102 and detector 104 form a set of projections 110. The source 102 and detectors 104 are translated to form a view at each of the angles. The views from each measurement angle are then combined to form an image of the object.

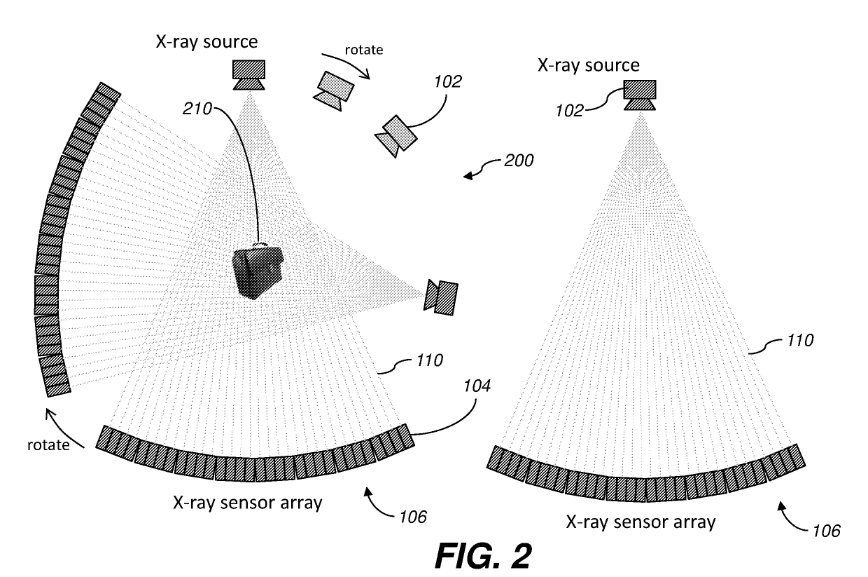

FIG. 2 shows a diagram of an example a second-generation fan-beam CT scanner 200 according to one embodiment. The scanner 200 is similar to that of FIG. 1, except an array 106 of detectors 104 is provided in a fan-beam pattern to allow for simultaneous imaging by the detectors to form a view. Again, each view includes a set of projections 110 formed by the X-ray source 102 and the array 106 of detectors 104. The source 102 and detectors 104 are then rotated about the object 210 (alternatively, a person, or other scan target) to form a view at a set of angles. A single view corresponds to a set of measurements from all the elements of the array at approximately a single instant of time. Each detector in the array is known as a channel, so a single view corresponds to a set of measurements from different channels. The objective is then to take this set of views and reconstruct a cross-section of the object 210 being imaged. Typically, the data collected from the scanner is organized into a sinogram. For a 2D scan, the sinogram is a 2D data structure indexed by the channel and view of each measurement.

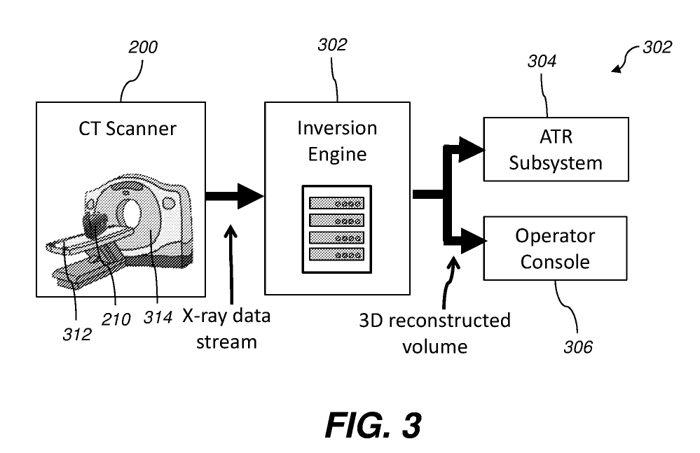

FIG. 3 shows a diagram of a computed tomography (CT) scanner system 300. The system may include the scanner (such as scanner 200), inversion engine 302, automatic target recognition (ATR) subsystem 304, and operator console 306. The scanner 200 includes the rotating gantry containing an X-ray source 102 and detector array 106 as discussed above. The inversion engine 302 is typically a system comprised of one or more computers together with software and associated communication peripherals that process the data from the scanner to produce a two or three-dimensional reconstruction of the object being scanned. In some embodiments, the reconstruction is rendered onto a computer display to be viewed by a human. In other cases, the two or three dimensional reconstructed image is communicated to another computing system that may detect, classify, and/or segment components of interest from the full two or three-dimensional volume. We refer to such a computing system herein as an ATR. In some cases, the reconstructed image is both viewed by a human and analyzed by an ATR system. It shall be understood that the CT scanners 100 (FIG. 1) or 200, the inversion engine 302, the ATR subsystem 304, and the operator console 306 may comprise one or more computer processing units (CPUs) operatively connected to multiple types of computer memory as described herein.

The scanner typically has a translating table 312 that contains the object 210. The table is designed to move through the rotating gantry 314 so that a series of slices can be measured. In some embodiments, the scanner 200 includes multiple rows of detectors as described above. In the illustrated example, the scanner 200 is referred to as multi-slice since it can measure projections through multiple slices in a single rotation of the gantry. In other embodiments, the object 210 being scanned passes through the gantry 314 as it rotates, in which case the scanner 200 is referred to as a helical scanner since the effective trajectory of the scan is a helix. In still further embodiments, the scanner may have no gantry and may instead comprise a series of X-ray sources at different locations that can be multiplexed to make a series of projection view measurements.

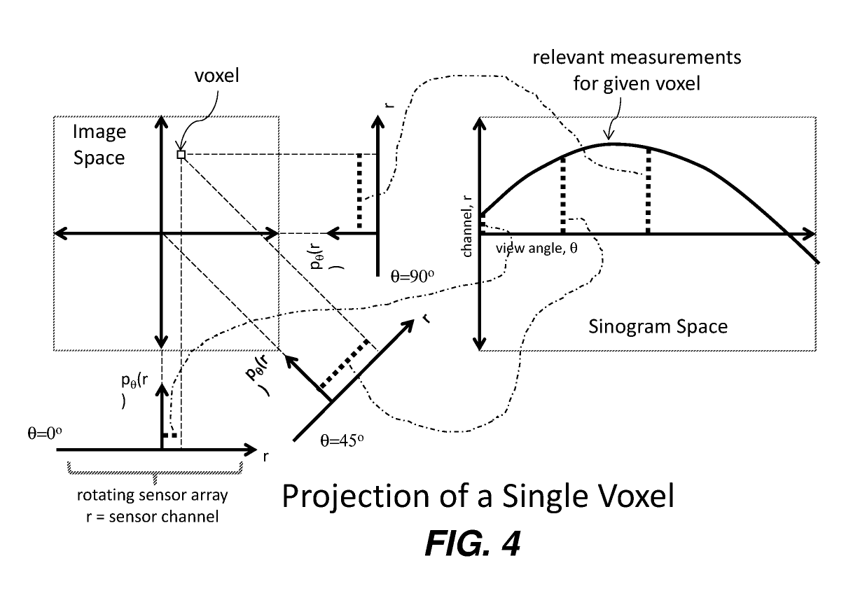

Referring to FIG. 4, a 2-D slice image of a 3-D object (such as object 210) is viewed from the side to provide one projected column of data at each angle. The projections, arranged as a function of angle, form a sinogram. If the object is one dot, the dot traces a sine-wave pattern in the sinogram. Data from multiple slice images can then be combined to form a 3-D image of the object. In FIG. 4, the X axis is the view angle and the Y axis is the displacement from the center of rotation of the detector 104. A “Channel” can be, e.g., an imager pixel in a 1-D CCD or CMOS image sensor array.

The sinogram can be stored in memory, e.g., RAM, in either row-major or column-major order format. In row-major order, consecutive elements of the rows of the array are contiguous in memory. In column-major order, consecutive elements of the columns are contiguous. In row-major format, consecutive elements in memory correspond to different view angles at the same channel whereas in column-major format, consecutive elements in memory correspond to different channels at the same view angle. When row-major format is used, the “fast variable” is the view index whereas when column-major format is used, the “fast variable” is the channel index.

Recent research has demonstrated great potential for MBIR to improve the performance of X-ray CT-based imaging in a wide range of applications including explosive detection systems (EDS), medical imaging, scientific imaging, and materials imaging. MBIR works by computing a 3D volumetric reconstruction of an object using a cost-optimization framework that takes into account a model of the scanner geometry and noise characteristics, as well as a model of the 3D image statistics.

Some MBIR systems permit reductions in X-ray dosage for a variety of clinical applications including lung cancer screening (˜80% reduction) and pediatric imaging (30-50% reduction).

In systems where dosage reduction is not as important, such as EDS scanners, MBIR offers other advantages:
1. Reduced Artifacts—MBIR can reduce artifacts such as streaking due to metal. This streaking is typically caused by a combination of un-modeled scatter, beam hardening, and photon-starvation. Reduced streaking can reduce missed detections and false alarms in EDS applications.
2. Improved Resolution—MBIR can improve resolution by combining a better scanner model with a better 3D model of image structure. Improved resolution can reduce missed detections and false alarms by improving object separation.
3. Novel Scanner Architectures—MBIR enables non-traditional scanner architectures by allowing for reconstruction with sparse view sampling. Traditional CT scanners require that the view angles be sampled densely at the Nyquist rate, which typically corresponds to the number of pixels (i.e., channels) across a view. By relaxing this requirement, it is possible to design novel CT systems without the need for a rotating gantry. This in turn has many applications in baggage and cargo scanning

Therefore MBIR offers the potential to a) reduce cost by reducing false alarm rates, b) extend EDS effectiveness in new threat scenarios, and c) enable the design of novel scanner hardware to meet emerging DHS/TSA needs.

To realize this potential, the image reconstruction speed of MBIR must be improved beyond that of currently available systems. Table 1 presents statistics collected for a conceptual EDS system design. The numbers and simulations are based on realistic models of scanner geometry and assumed 3D image phantoms.

TABLE 1
Measured Performance on Simulated EDS Application

Algorithm: NHICD
Hardware: Multicore CPU
Parallelization: 1 core
Number of Data Sets: 21
Average number of iterations to convergence (Equits): 3.69
Maximum RMSE at termination: 8.87 HU
Spatial resolution: 512 × 512 [symbol] n = 262,144
Average reconstruction time per slice per view: 63.8 msec/(slice * view)

Based on the data in Table 1, Table 2 estimates the performance on a single processor core implementation for a full 3D reconstruction, and determines the speedup required for an implementation of MBIR in a real-world EDS application. Assuming a 15 sec processing time per bag (i.e., real-time processing with 240 bags per hour), then this implies that the required speedup is approximately 900:1, a metric which to date has limited the adoption of MBIR to EDS applications.

TABLE 2
Estimated Baseline Performance on Simulated EDS Application

Algorithm: NHICD
Hardware: Multicore CPU
Parallelization: 1 core
Average number of iterations to convergence (Equits): 3.69
Spatial resolution: 512 x 512 [symbol] n = 262,144
Number of slices: 400
Number of views: 512
Average reconstruction time per slice per view: 63.8 msec/(slice * view)
Total reconstruction time for 3D volume:
$13 \text{,} 066 sec = ( 63.8 \frac{msec}{slice * view} ) \times ( 400 slice ) \times ( 512 view )$
Required computational speedup: 871: 1 = (13,066 sec)/(15 sec/bag)

MBIR is based on the numerical solution of an optimization problem described by,

$$\hat{x} = arg {min}_{x \geq 0} \{ {‖ y - Ax ‖}_{D}^{2} + u ( x ) \} \qquad ( 1 )$$

where y is an m-dimensional vector containing the measured CT data, x is a n-dimensional vector containing the reconstructed image, A is an m×n-dimensional matrix containing the forward model of the scanner geometry, D is an m×m-dimensional diagonal matrix containing the inverse variance of the scanner noise statistics, and u(x) is the regularizing prior model used to incorporate knowledge of image behavior. Each element of x then represents the value of a volume element, or voxel. In practice, x and y can be very large, so computing the solution to the optimization problem can be a formidable task.

Broadly speaking, there are two general approaches to solving this large inverse problem.
1. Simultaneous methods—Simultaneous methods are based on the simultaneous computation of Ax and A^{t}y as the kernel of the numerical optimization approach. These methods include gradient descent (GD), conjugate gradient (CG), preconditioned gradient and conjugate gradient descent (PGD, and PCG), and ordered subset (OS) methods.
2. Coordinate descent methods—Coordinate descent methods are based on sequential optimization with respect to individual voxels in the image. These methods include Iterative Coordinate Descent (ICD), Non-Homogeneous Iterative Coordinate Descent (NHICD), and grouped coordinate descent (GCD).

Simultaneous methods are optimization approaches which are widely used. However, they have a number of disadvantages for MBIR. Traditional GD has very slow convergence and may require hundreds of iterations to converge. Hence in order to speed convergence, such methods must be combined with processes such as preconditioning and ordered subsets. Simultaneous methods, while potentially promising, have the disadvantage that preconditioners require very sophisticated and difficult custom algorithmic tuning for each CT system. This imposes a great limitation on the commercial potential and economies of scale for simultaneous methods since each reconstruction engine must be custom designed for its corresponding specific CT imaging system.

Ordered subset methods may also be combined with preconditioning, and are also a key ingredient to speed convergence. Ordered subset methods work by grouping views into subsets so that each sub-iteration can be much faster. Domain decomposition methods offer the potential for O (n log n) computation of simultaneous projections (Ax) as opposed to the direct O(n^{3/2}) complexity. This is a potentially huge speedup for real systems in which one might have n≈10⁸. However, domain decomposition methods require a full set of views, so they do not work well when combined with ordered subset methods or with CT systems that use sparse views. This severely limits the application of domain decomposition methods.

Alternatively, ICD and NHICD optimization methods have been shown to exhibit very rapid and robust numerical convergence for a wide variety of MBIR applications spanning a wide range of geometries, noise statistics, and image models. Intuitively, coordinate descent methods are fast and robust because each pixel is updated to best minimize the cost function, so there is tight feedback in the optimization process. In practice, NHICD has been shown to converge in 3 to 6 iterations. Also, when properly implemented on older computer processing unit (CPU) architectures it has been demonstrated that ICD can use the hardware resources very efficiently, so the actual Wall-Time Per Iteration (WTPI) is low and comparable to that of the best implementations of simultaneous methods. So, the rapid convergence is a decisive advantage of coordinate descent methods on multi-core platforms. However, a disadvantage of ICD is that it requires that memory be accessed along columns of the A matrix from equation (1). On GPUs with very wide buses and other specialized hardware, this constraint restricts the order of memory access, and can slow the theoretical WTPI of coordinate decent methods. Memory access can also be problematic on more modern CPUs with advanced multi-core architectures that may incorporate parallel computation into their design. For example, a modern CPU may be capable of computing 32 floating point multiply accumulate operations per core in a single clock cycle. Keeping all processing hardware fully employed has become increasingly more difficult as CPU and GPU architectures have become more sophisticated.

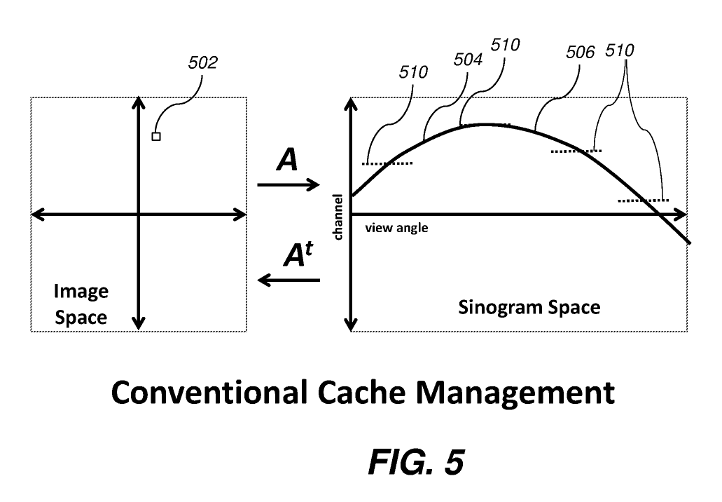

FIG. 5 shows an example of how memory access occurs in a typical implementation of a coordinate decent algorithm such as ICD. First the measured data is read from the scanner or other device, preprocessed, and stored into a 2 dimensional array of memory known as the sinogram space. Preprocessing may comprise a number of steps including normalization, logarithms, and polynomial corrections which are well known in the art. In this case, the sinogram space is a 2 dimensional array of data in row or column major format. The weight sinogram may also refer to associated weights which specify how reliable each measurement is. The error sinogram may also store the values e=y−Ax which equal the difference between the measured sinogram and the projection of the image. We will use the term sinogram to refer to any of these forms of data. In addition to storing the measured data in the sinogram space, memory must be allocated to store the image that will be reconstructed from the measured data. This allocated memory is denoted as the image space in FIG. 5. The allocated memory for the image space can be initialized in wide variety of ways. It can be initialized as a constant value. It can be initialized using a reconstruction algorithm such as filtered-back projection (FPB), or it can even be initialized using another iterative reconstruction algorithm. Regardless of how it is initialized, the error sinogram must be initialized to be consistent with the initial image so that e=y−Ax where y is in the measured and preprocessed data.

Once the image space and sinogram data and error sinogram are initialized, a coordinate descent algorithm such as ICD computes the reconstruction by computing a series of voxel updates until convergence occurs. In order to update a single voxel 502 it is necessary to access the voxel data 504 and then use this data to compute an update of the voxel. After the voxel is updated, it is also necessary to update the error sinogram so that the relationship e=y−Ax is maintained. In practice, many voxel updates must occur in order to compute a single reconstructed image.

FIG. 5 illustrates a key technical challenge in speeding up ICD on computing platforms that meet commercial cost constraints. The core computational operation in ICD optimization is the “voxel update” in which the value of a single voxel is updated to minimize the total cost in Equation (1). The voxel update operation requires access to a set of measurements stored in the “sinogram space”. The sinogram is the name given to the data streamed from the scanner's detectors 104 into memory. It is given this name because when the data is visualized as an image, it appears to have sine wave patterns in it that are generated by dense objects in the image. The sinogram is a 2D array of data that contains the measurements from the scanner 100 or 200 indexed by the channel and the view. In FIG. 5, the sinogram is shown with the channel along the vertical axis and the view along the horizontal axis. Notice that a single voxel 502 in the image is measured by a set of projections in the sinogram that trace out the path of a sine wave 506. We refer to the data that corresponds to a specific voxel 502 as the voxel data 504. It is this voxel data 504 that forms the sine wave shape 506. The amplitude of the sine wave 506 corresponds to the distance of the voxel from the center of rotation, and the phase of the sine wave 506 corresponds to the angle of the voxel 502 in polar coordinates. In order to update the value of a voxel 502 using the ICD algorithm, it is necessary to access the voxel data 504. For the measurement data sinogram and weights sinogram, this typically requires reading the voxel data. For the error sinogram, this also requires writing to the sinogram so that the consistency condition e=y−Ax is maintained after the voxel update.

In a modern high-performance computer or GPU, data to be processed is read from the main memory into a series of specialized cache memories before being processed. This is done because the cache memories are much smaller but also much faster than the main memory. In some cases, there is a series of ever smaller and faster cache memories. For example, an L1 cache memory is typically the fastest and smallest cache memory. By fast memory, we typically mean that the cache memory has both lower latency to read and also can provide more data in a fixed amount of time. The processor may also have L2 cache that is larger and slower than L1 cache, but still smaller and faster than main memory. There may also be L3, and L4 caches and so on. If a processor goes to read a single data entry in the main memory, it first checks to see if the data entry is in the L1 cache. If it is in the L1 cache, it uses this value. If it is not in the L1 cache, then the processor hardware reads a cache line of data from the main memory. A cache line corresponds to a fixed predetermined number of data values or bytes that is transferred from the main memory to the cache memory. These cache lines also start at predetermined locations in the main memory. Both the length of cache lines and their predetermined starting locations are fixed for each specific processor and typically cannot be changed. The key issue is that to read one byte of data from main memory it is required that the entire cache line containing that byte of data be read into the cache memory.

In FIG. 5, the sinogram elements, referred to herein as voxel data 504, required for a voxel update are shown as a line with a sinusoidal shape. If the sinogram is stored in row major order, then due to the organization of hardware, the memory cache lines of the computer processor being used by the system must have the form of the straight lines 510 shown in FIG. 5. Now in order to read or write to the voxel's data, it is necessary to read or write each element of the voxel data. This in turn requires that the associated cache line be read that contains that data. In FIG. 5, notice that the cache lines 510 typically intersect with the voxel data 504 at only a single point. This means that the majority of the data that is brought in into cache memory with the cache line 510 is of no value in performing the voxel update. Alternatively, the voxel data are not typically adjacent to one another in the physical memory of the system. This has a variety of negative effects on the performance of the optimization. First, cache memory (typically on-processor memory that can be accessed 100 times or more faster than system RAM, but which may also be located outside of the processor) is not utilized efficiently. There are often two orders of magnitude or more of main memory (e.g., system RAM) than cache memory. Thus, cache memory cannot typically hold the entire memory used in the computation, and because of the slow speed of system RAM it is expensive to repeatedly bring data from RAM into cache. For this reason it is desirable that, when a block of data (referred to as a cache line 510) is brought into a cache memory, as many words as possible in the cache line be operated on as many times as possible before the cache line is evicted from the cache memory to make room for other data. As illustrated in FIG. 5, this standard prior art implementation of ICD will waste the majority of the data read from main memory, so the effective rate of data transfer will be a fraction of the rate that is theoretically possible. Therefore, the CPU will become starved for data, and it will spend the majority of its time waiting for data.

Accelerators, such as graphical processing units (GPUs), have local memory that can be viewed as a cache memory that is managed by the processor that controls it. For data to be efficiently transferred into this logical cache memory, large blocks of data should be located contiguously in memory. Therefore, in the graphic of FIG. 5, the effective size of a cache line 510 is even longer than it is for a multicore processor cache. Thus, having algorithms that place data associated with a voxel 502 contiguously in memory rather than distributed in a sinusoidal pattern is at least as important for GPUs as it is for multicore processors.

From FIG. 5 it can be seen that the cache lines 510 are not well aligned with the sinusoidal voxel data 504 of memory that must be accessed for MBIR updates, and a failure to exploit cache memory or GPU local memory will result in a processor spending most of its time waiting for data to come from memory. Consequently, prior art implementations of ICD can have poor processor utilization in practical systems which utilize cost effective commercially available processors.

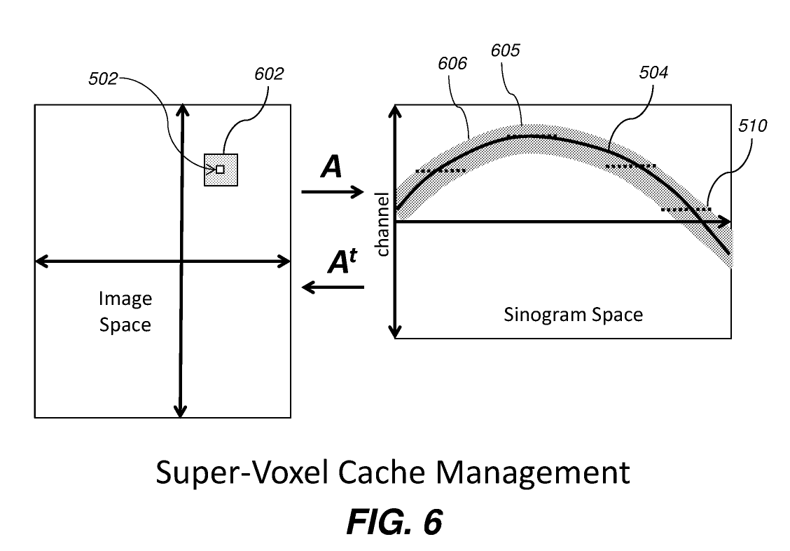

According to one embodiment of the present disclosure, the scanner system 200 is configured to group voxels into “super-voxels”. As shown in FIG. 6, a super-voxel (SV) 602 includes of a plurality of voxels 502 in the image space that are clustered together in a region in the image. In further embodiments, a super-voxel may include any set of voxels that are substantially local or substantially adjacent in 1, 2, or more dimensions in the image. Each individual voxel 502 in the super-voxel 602 then corresponds to a set of sinogram memory entries, such as those shown in FIG. 4. Because the voxels 502 in the super voxel 602 are spatially localized in the image, corresponding memory entries (e.g., in the sinogram space) for each voxel substantially overlap. The union of all sinogram memory entries corresponding to all the voxels 502 in the super-voxel 602 then typically forms a wide sinusoidal band 605 of memory as shown in FIG. 6. The width of the band 605 does not need to be constant. This band 605 of memory is referred to herein as the super-voxel data 606. The super-voxel data 606 includes all of the memory that must be accessed to update the voxels 502 in the super-voxel 602.

FIG. 6 shows a typical super-voxel 602 along with its associated super-voxel data 606. It also shows a single voxel 502 in the super-voxel 602 and its associated voxel data 504. In order to update a super voxel 602, either serial or parallel updates of its constituent voxels 502 are performed. In order to update the voxel 502, it is necessary to read and/or write to the voxel data 504 in the sinogram. When this is done, the process reads all cache lines 510 that are required to access every data element in the voxel data 504. These cache lines 510 contain more data than is required for the update of the voxel 502. However, the cache lines 510 typically substantially overlap with the super-voxel data 606. This means that a good portion of the super-voxel data 606 will end up in cache memory after the voxel 502 is updated. Consequently, subsequent voxel updates in the super-voxel 602 will be much faster since the required data is already in cache memory. After all voxels 502 are updated in the super-voxel 602, the entire super-voxel data 606 will have been moved to cache memory along with some additional data that is not required but near the super-voxel data 606 in the sinogram. However, the relative fraction of data that is read into cache memory, but not required, will be much smaller than in the case of FIG. 5. In other words, more of the retrieved data will be utilized for processing, resulting in less waste and increased efficiency.

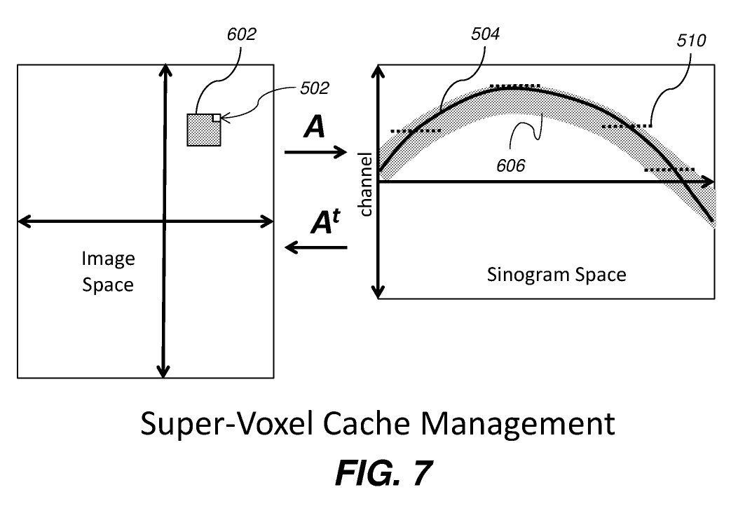

FIG. 7 shows the special case when a voxel 502 is updated in the super-voxel 602 that falls near the edge of the super-voxel 602. In this case, the voxel data 504 will be nearer the top of the super-voxel data 606 location at some views and near the bottom of the super-voxel data 606 location at other views. Notice that in this case, the cache lines 510 sometimes fall outside of the super-voxel data 606. These values that are read into cache memory from outside the super-voxel data 606 location are wasted. However, the relative waste for this case is much smaller than the waste in FIG. 5.

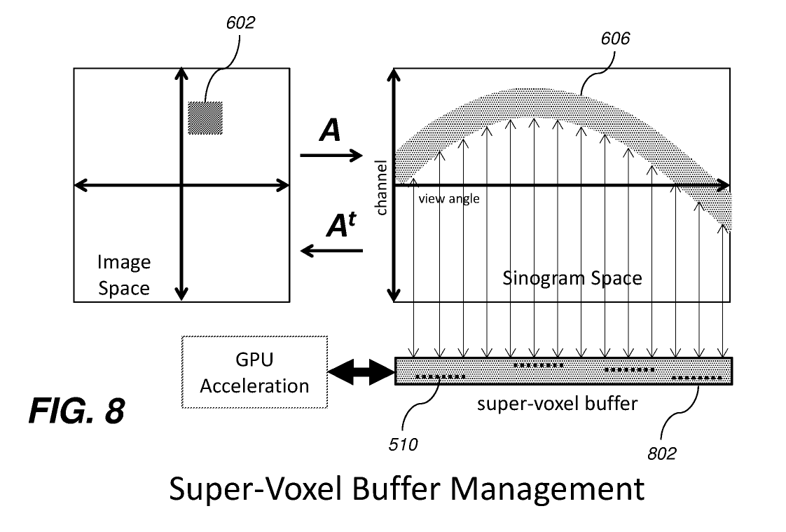

FIG. 8 shows a further improvement on the super-voxel scheme with incorporates a super-voxel buffer (SVB) 802. When the super-voxel buffer 802 is used, the data is copied typically within main memory from the super-voxel data 606 memory location to the SVB. In this case, column major order is typically used so that data can be rapidly copied from the super-voxel data 606 to the SVB 802. Once this is done, all further processing required for the super-voxel update is performed using the SVB 802, and once the super voxel update is completed, then the SVB 802 is optionally copied back to the super-voxel memory 606 in the sinogram space. The advantage of the SVB 802 is that all the voxel data in the SVB 802 is much straighter than it was in the original sinogram space. In this case, straighter means that the voxel data falls substantially along either rows or columns of the SVB 802 depending on whether row major or column major ordering is used. If column major ordering is used, then accessing along rows of the SVB 802 corresponds to accessing data values at fixed stride. Fixed stride means that data values are separated by a fixed number of bytes. Modern processors often have specialized hardware (such as prefetching hardware) to make access of data along fixed strides faster. If the row major ordering is used, then accessing along rows of the SVB corresponds to accessing data values that are adjacent in memory. This is illustrated in FIG. 8 where cache lines 510 fall along rows of the SVB 802. In this case, access to the SVB 802 is very fast since modern processors are typically optimized to access cache lines 510 corresponding to sequences of adjacent data values in memory.

Once used, the SVB 802 is typically automatically transferred to the processors cache memory, so repeated updates of the individual voxels in the super-voxel may be computed very efficiently and at very high speed. Therefore, multiple updates of voxels 502 in the super-voxel 602 can be computed very rapidly until the desired level of convergence of that MBIR algorithm is achieved on the super-voxel 602. Each update of a voxel 502 is typically designed to reduce or minimize the cost function of equation (1). This can be done using a variety of well known methods to one skilled in the art. These methods for updating the voxel 502 require access to the voxel data 504, which typically comprises the associated sinogram measurements, weights, and errors. Then, once updates of voxels 502 in the super-voxel 602 are complete, the super-voxel buffer 802 can be copied back to the sinogram memory. This super-voxel update is then performed repeatedly across the image until total convergence of the reconstructed image is achieved.

According to further embodiments, multiple super-voxel buffers 802 may be created and processed in a pipelined manner to further improve system efficiency. In different embodiments the super-voxel buffer 802 may be physically realized as different cache memories in a processor's cache memory hierarchy, RAM memory, GPU RAM memory, or special memory added to the computer processing system. In some cases, the sinogram memory can be directly copied to an explicitly defined super-voxel buffer section of memory. This is referred to herein as an explicit super-voxel buffer. In other cases, software commands can be used to cause the existing memory control system of the hardware to copy the data of the super-voxel buffer 802 into cache memory or other fast memory. This is referred to herein as an implicit super-voxel buffer. Various examples use corner-turning memory or GPU 2-D mapped memory.

The mapping from the sinogram to the super-voxel buffer 802 can be determined from the forward matrix A. This is important since it means that the super-voxel buffer method can be implemented without custom tuning based on the system geometry. This means that the same general purpose inversion engine 302 can be used to solve a wide range of inversion problems for different applications by simply loading the system matrix, A, and the parameters of the prior model u(x).

Table 3 illustrates the effectiveness of the above described super-voxel method on a specific CT reconstruction problem. Table 3 lists processor efficiency and execution time for the baseline algorithm and the super-voxel algorithm. In this problem, we directly compared the super-voxel MBIR reconstruction approach to an equivalent benchmark implementation of MBIR without the super-voxel method. In the particular example, the processor efficiency and execution time was measured for a reconstruction problem with the following parameters: Image size: 512×512, number of slices: 1, number of view angles: 720, number of channels: 1024. Each method was run to approximate convergence with an approximate average of 4 updates per image voxel. The efficiency measures the percentage of floating point operations per second as compared to the theoretical maximum for the processor. Here we see that the super-voxel method results in a speedup of approximately 22× in efficiency and 25× in time.

TABLE 3

| | Processor efficiency | Execution time |
|---|---|---|
| Benchmark MBIR | 0.3% | 253 sec |
| Super-voxel MBIR | 6.6% | 10.0 sec. |
| Performance improvement | 22x | 25.3x |

The super-voxel algorithm can be implemented in further embodiments to work in various ways that are described in more detail below.

For example, in one embodiment, updates within a super-voxel 602 can be done in parallel. In this case, ICD voxel updates in the super-voxel 602 are computed independently. While the original ICD algorithm assumed sequential updates, parallel updates may be desirable since they allow a large number of processing elements in the hardware to be used. In addition, the update of a single voxel 502 can be parallelized by assigning multiple processing elements to different views or regions of the sinogram or SVB. Also, parallelization can be achieved in multiple ways by using both parallel voxel updates and parallel sinogram computations.

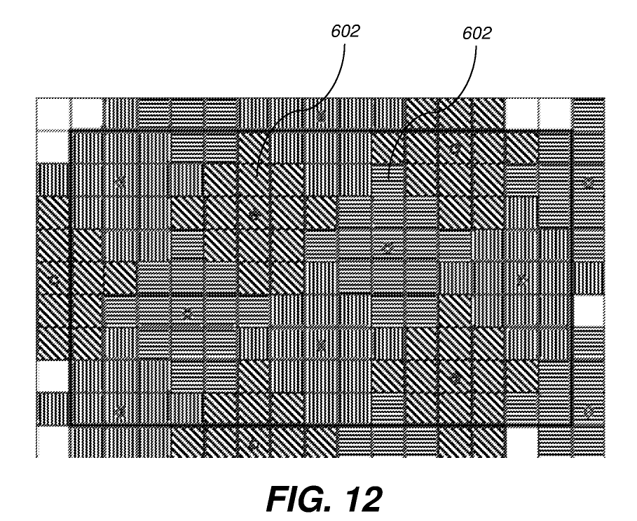

Because the size of a super-voxel 602 affects both the processing speed and convergence of the algorithm, in some examples super-voxels 602 have a uniform size and all voxels 502 are contained in a super-voxel 602 of this uniform size. Some embodiments include techniques to spatially pack voxels and update them in non-uniform sequences in order to speed convergence. FIG. 12 illustrates an example of how the super-voxels 602 can be packed in 2D. Different hatching patterns distinguish adjacent super-voxels 602. Similar techniques are possible for 3D packing. In order to achieve the most rapid convergence with the minimum computation, it may be desirable to have a small amount of overlap between adjacent super voxels 602. Also, it may be desirable to have two sets of super voxels 602 that fully partition the image with each set, or phase, being offset by ½ of a super voxel 602 in both the horizontal and vertical directions.

In some examples, to maximize cache efficiency, each super-voxel 602 may be substantially round or hexagonal so that the width of the super-voxel buffer 802 is minimized. In general, the best super-voxel shape represents a tradeoff between these the two competing needs of minimizing the size of the super-voxel data and minimizing overlap of the super-voxels. In practice, hexagonal super-voxels can pack optimally in 2D while providing nearly spherical shape. In addition, super-voxels can be visited in non-sequential order so that more important super-voxels 602 are visited more frequently. In some cases, the same super-voxel 602 can also be updated two or more times in a row. This can be particular useful if a particular super-voxel 602 has a large change in its first update. Both optimized packing and non-sequential updates can substantially improve convergence speed and reduce computation. In some cases, the updating of a super voxel 602 or a voxel 502 in the super-voxel 602 can be skipped. This is particularly desirable if the all the voxels 502 in the super-voxel are zero or if the voxels surrounding a particular voxel are zero since this can be a strong indication that the voxel or super-voxel have already converged to their final value.

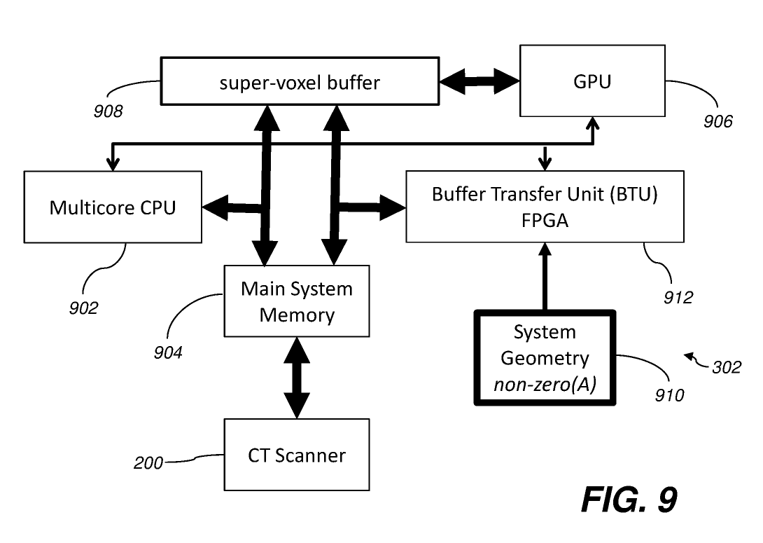

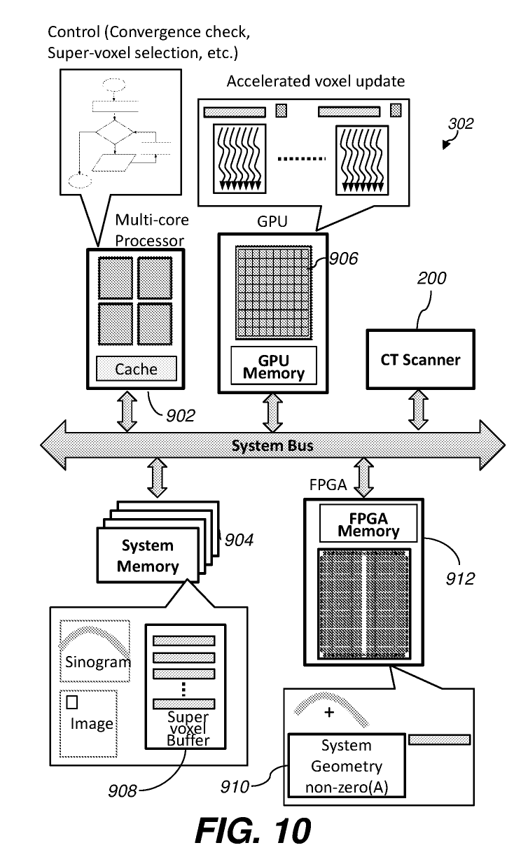

FIG. 9 shows a sample hardware architecture of the inversion engine 302. As shown, the inversion engine 302 may comprise a single or multicore CPU 902, a main system memory 904 (which is in communication with the CT scanner 200), GPU 906, super-voxel buffer 908, system geometry unit 910, and buffer transfer unit/FPGA 912, which may be operatively connected by a system bus. The CPU 902 and the GPU 906 may include cache memory or other forms of high-speed access memory. FIG. 10 shows a further example architecture for the inversion engine 302, with the single or multicore CPU 902, main system memory 904 (which is in communication with the CT scanner 200), GPU 906, super-voxel buffer 908, system geometry unit 910, and buffer transfer unit/FPGA 912, which may be operatively connected by the system bus.

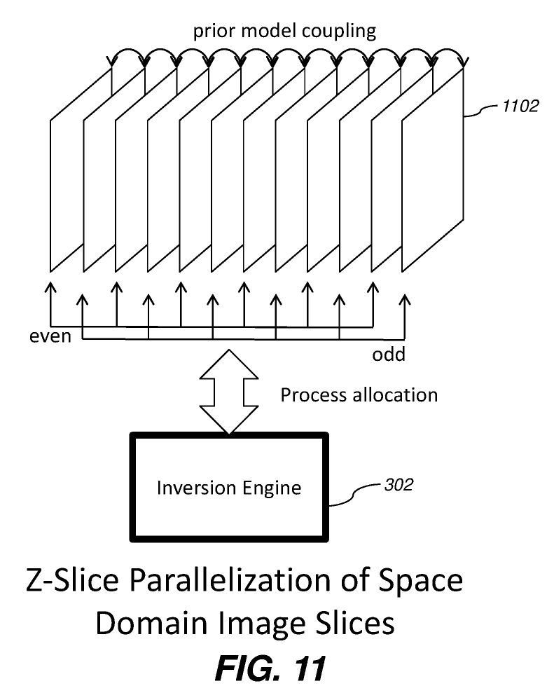

FIG. 11 illustrates how parallelization may be exploited across reconstructed image slices 1102 in the z-axis. For 3D reconstruction of axial CT data, the degree of parallelization is very high across slices. In fact, if the parallelization is organized as interlaced groups of even and odd slices 1102 as shown, the associated processes are completely decoupled in most practical cases. Thus, in a 400-slice reconstruction, at least 200 slices can be processed in parallel with little or no communication required between processes.

According to one embodiment, four levels of parallelism are listed below, any of which may be implemented according to the present disclosure. The forms of parallelism are listed from finest grain to coarsest grain. So the fine grain parallelism is best exploited by computational hardware that is tightly coupled, whereas the course grain parallelism is best exploited by loosely couple hardware.
1. Intra-voxel parallelism: Data within a super-voxel buffer 802 may be processed in parallel in order to speed the update of a single voxel 502.
2. Intra-SV parallelism: Multiple voxels 502 within a single super-voxel 802 may also be updated in parallel. This simultaneously update of multiple voxels 502 in a super-voxel 602 is referred to herein as Intra-SV parallelism.
3. Inter-SV parallelism: Multiple super-voxels 602 may also be updated in parallel. This type of parallelism is particularly useful when dividing processes across different cores in a multicore processor. In order to implement this type of parallelism we introduce a novel data structure, which is referred to herein as an augmented SVB. This is described in more detail below.
4. Inter-slice parallelism: Different slices of the 3D volume can be updated in parallel. We refer to this as Inter-slice parallelism. This can be very important in splitting computation across different nodes in a computing cluster.

Inter-SV parallelism (with each SV 602 containing more than one voxel 502) has fewer conflicts on shared data than intra-SV parallelism, reduced communication overhead and sufficient work to keep every core busy. Intuitively, fewer conflicts mean that inter-SV parallelism allows each core to have more work to do before a possible multicore communication. In addition, the frequency of communication is also reduced proportionally to the SV 602 size.

Moreover, these four major levels of parallelism are largely orthogonal, so that the total number of parallel operations increases as the product of the number of parallel threads in each level. This is important because the number of cores in modern computing systems is rapidly increasing with each new generation of device. Hence, keeping all these cores efficiently busy is a crucial requirement for fast efficient MBIR. In addition, it is also important to note that these four levels of parallelisms are hierarchical since one slice comprises a plurality of SVs 602 and each SV 602 comprises a plurality of voxels 502.

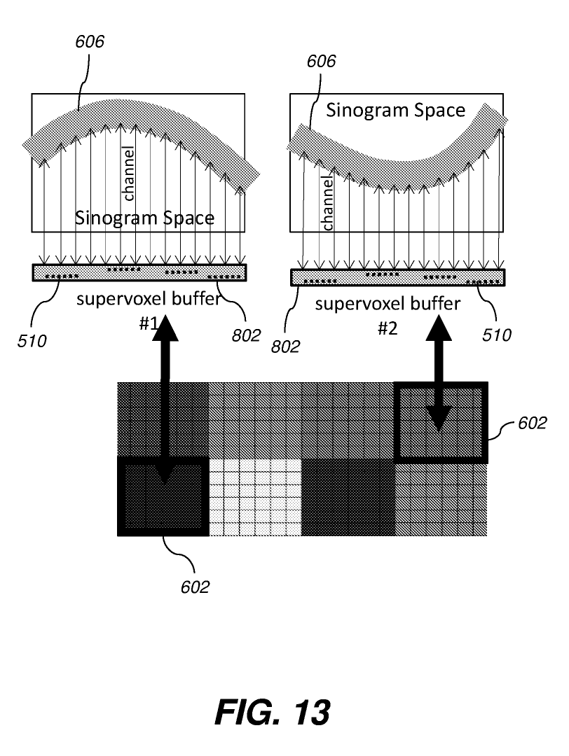

For Inter-SV parallelism, we operate on multiple SVs 602 that are far apart in the image space. The distance between these SVs 602 also minimizes interference and lock contention resulting from simultaneous SVs update in CT scanning systems. This is because when super-voxels are further apart, then the amount of overlap in the sinogram space is smaller. This means that there is less potential conflict when updating the sinogram data from two different SVBs. As shown in FIG. 13, each SV 602 has its own SVB 802, and within a SV 602 updates are performed sequentially or in parallel, ensuring good cache locality. This process using multiple SVs 602 and SVBs 802 is referred to herein as Augmented SVB. In the beginning of the process, each computer processor core is typically assigned one SV 602 and then each core creates its own SVB 802, denoted SVB^{k} for the k^{th} SV 602. The augmented SVB, enables inter-SV parallelism by keeping two copies of the SVB 802, with one copy containing the initial value of the SVB 802 and the second copy containing the updated value of the SVB 802. The augmented SVB is a key innovation that makes Inter-SV parallelism possible because it allows the full sinogram to be properly updated even when different SVBs 802 contain data from the same region of the sinogram.

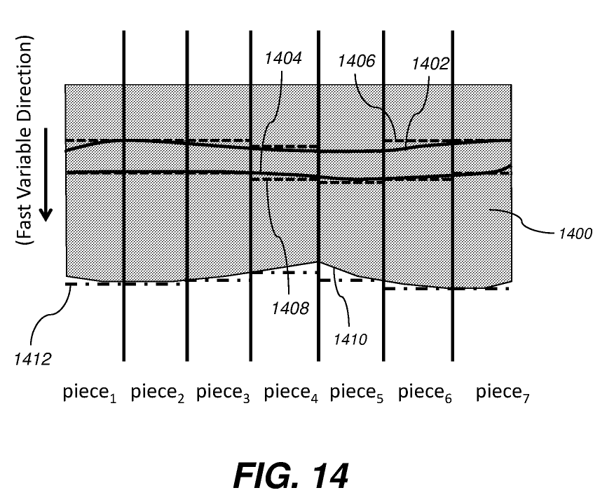

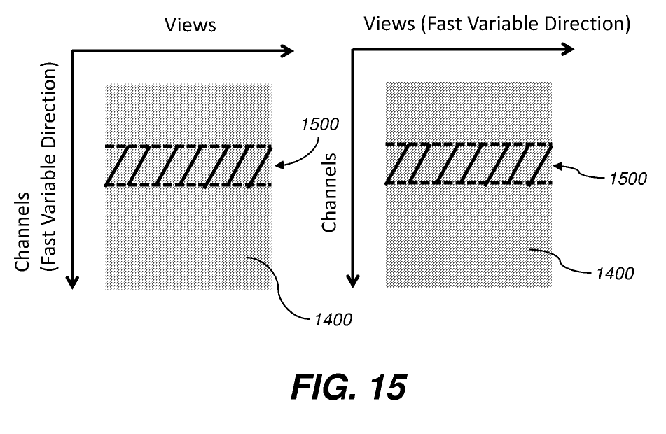

FIGS. 14 and 15 illustrate the piece-wise transposed SVB according to one embodiment. FIG. 14 shows an SVB 1400 corresponding to a particular super-voxel. The thin region bounded by two solid lines 1402 and 1404 shows the upper and lower channel numbers of the voxel data that are accessed for the update of a single voxel in the super-voxel. Notice that this region is not perfectly straight and curves up and down slightly as a function of view. This curving makes the memory access somewhat irregular, which can greatly reduce access speed of the processor's cache memory since the access stride is not constant. In order to make the access patterns more regular, the SVB is first broken into pieces (illustrated here as pieces 1-7). Notice that the height of the SVB 1400 is not exactly constant so the bottom edge 1410 of the SVB 1400 is somewhat irregular. In order to make the SVB 1400 more regular, the bottom of each piece of the SVB is padded with zeros on the bottom to make its height constant. The dot-dash line 1412 indicates the bottom of each constant height piece. For each of these pieces, the dotted straight lines 1406 and 1408 indicate the upper and lower bounds of the curved lines 1402 and 1404. The entries of the A matrix are padded with zeros so that a slightly larger region can be processed corresponding to these dotted lines 1406 and 1408. The advantage of the dotted regions is that the regions are bounded by perfectly straight lines, which makes memory access much faster.

FIG. 15 shows the next very important step of transposition for each piece 1-7 of the SVB 1400. The SVB 1400 is a 2-D array, so it must be stored in the computer's memory as a 1-D array in raster order. There are two options for raster ordering of a 2-D array. The first option is known as column major ordering in which each column of the array is stored in order. In this case, the channel index is the fast variable. The second option is known as row major ordering in which each row of the array is stored in order. In this case, the view index is the fast variable. Initially, the channel variable is the fast variable in the SVB since this allows faster transfer of memory from the sinogram to the SVB 1400. However, this also means that crosshatched area 1500 in FIG. 15 is broken into disjoint pieces in the computer memory. These broken memory pieces can greatly slow down access to the required memory for a single voxel's update. The piece-wise transposition transposes the data in each piece of the SVB 1400 so that the view variable becomes the fast variable. After transposition, the crosshatched region 1500 that must be accessed for the update of a voxel represents a single contiguous array of entries in the computer's memory. Therefore, this array of values can be accessed very rapidly from the processor's memory or cache.

Table 1 shows the results of applying the SV-ICD and the parallel SV-ICD (PSV-ICD algorithms on a CPU multicore: 2 Intel Xeon ES-2660 v3 2.6 GHz processors with 20 cores using a standard tomographic data set [25,26]. The column labeled “Equits” represents the number of equivalent iterations required for convergence of the MBIR algorithm. The column labeled “Recon time” is the total time in seconds required to perform MBIR reconstruction, and the column labeled “speedup” shows the speedup resulting from SV-ICD or PSV-ICD for each case. Notice that with only one core the serial version of SV-ICD runs 55 times faster than the baseline ICD implementation of MBIR. However, the PSV-ICD algorithm allows for the use of multiple parallel cores and results in a total speedup of 496× when 20 cores are used.

TABLE 1
Speedup of MBIR reconstruction resulting
from SV-ICD and PSV-ICD algorithms.

| | Baseline | Serial | PSV-ICD | PSV-ICD | PSV-ICD | PSV-ICD |
|---|---|---|---|---|---|---|
| | ICD | SV-ICD | (1 core) | (4 core) | (16 core) | (20 core) |
| Equits | 4.0 | 4.0 | 4.0 | 4.1 | 4.1 | 4.2 |
| Recon time (sec) | 253 | 4.6 | 6.9 | 2.0 | 0.61 | 0.51 |
| Speedup | 1x | 55x | 36x | 126x | 414x | 496x |

Finally, it can be observed that the SV-ICD and PSV-ICD algorithms can be used to compute the conventional forward and back projection without full implementation of MBIR. In this case, SV-ICD and PSV-ICD can be used to only compute the forward projection Ax or the back projection A^{t}y. This is important since the SV-ICD and PSV-ICD algorithms can then be used to implement either conventional FBP reconstructions, or other types of iterative reconstruction based on simultaneous updates. These other iterative reconstruction algorithms include but are not limited to gradient decent, preconditioned gradient descent, conjugate gradient, preconditioned conjugate gradient, simultaneous iterative reconstruction technique, algebraic reconstruction technique and variations of these using ordered subset techniques.

Steps of various methods described herein can be performed in any order except when otherwise specified, or when data from an earlier step is used in a later step. Exemplary method(s) described herein are not limited to being carried out by components particularly identified in discussions of those methods.

Various aspects provide more effective processing of CT data. A technical effect is to improve the functioning of a CT scanner by substantially reducing the time required to process CT data, e.g., by performing ICD or other processes to determine voxel values. A further technical effect is to transform measured data from a CT scanner into voxel data corresponding to the scanned object.

Various aspects described herein may be embodied as systems or methods. Accordingly, various aspects herein may take the form of an entirely hardware aspect, an entirely software aspect (including firmware, resident software, micro-code, etc.), or an aspect combining software and hardware aspects These aspects can all generally be referred to herein as a “service,” “circuit,” “circuitry,” “module,” or “system.”

Furthermore, various aspects herein may be embodied as computer program products including computer readable program code (“program code”) stored on a computer readable medium, e.g., a tangible non-transitory computer storage medium or a communication medium. A computer storage medium can include tangible storage units such as volatile memory, nonvolatile memory, or other persistent or auxiliary computer storage media, removable and non-removable computer storage media implemented in any method or technology for storage of information such as computer-readable instructions, data structures, program modules, or other data. A computer storage medium can be manufactured as is conventional for such articles, e.g., by pressing a CD-ROM or electronically writing data into a Flash memory. In contrast to computer storage media, communication media may embody computer-readable instructions, data structures, program modules, or other data in a modulated data signal, such as a carrier wave or other transmission mechanism. As defined herein, “computer storage media” do not include communication media. That is, computer storage media do not include communications media consisting solely of a modulated data signal, a carrier wave, or a propagated signal, per se.

The invention is inclusive of combinations of the aspects described herein. References to “a particular aspect” (or “embodiment” or “version”) and the like refer to features that are present in at least one aspect of the invention. Separate references to “an aspect” (or “embodiment”) or “particular aspects” or the like do not necessarily refer to the same aspect or aspects; however, such aspects are not mutually exclusive, unless otherwise explicitly noted. The use of singular or plural in referring to “method” or “methods” and the like is not limiting. The word “or” is used in this disclosure in a non-exclusive sense, unless otherwise explicitly noted.

The invention has been described in detail with particular reference to certain preferred aspects thereof, but it will be understood that variations, combinations, and modifications can be effected within the spirit and scope of the invention.

1. A tomography system, comprising:
one or more computer processors having a first memory, the first memory having a first access speed;
a second memory having a second access speed slower than the first access speed; and
one or more program modules stored on a third memory and executable by the one or more processors to perform operations comprising:
receiving measured data from a tomography scanner;
storing the measured data in the second memory; and
creating an initial image from the received measured data, the initial image comprising a plurality of voxels;
updating a super voxel in the initial image, the super voxel comprising a plurality of voxels whose corresponding data locations in the second memory substantially overlap, the updating performed by retrieving said corresponding data from the second memory and storing said corresponding data in the first memory, said corresponding data comprising a subset of the measured data.

2. The system according to claim 1, wherein the first memory is organized in cache lines, and the updating includes rearranging the portions of the measured data so that successive accesses to the measured data in the first memory by the at least one of the processors proceed sequentially along each of the cache lines.

3. The system according to claim 1 or claim 2, wherein the third memory is part of the first memory or the second memory.

4. The system according to any of claims 1-3, wherein the measured data forms a sinogram.

5. The system according to any of claims 1-4, the operations further including storing the measured data in a memory buffer, the memory buffer located in the first memory or the second memory, the measured data corresponding to the super voxel.

6. The system according claim 5, wherein the memory buffer is accessed using fixed stride retrieval.

7. The system according to claim 5, wherein the memory buffer is transposed to eliminate gaps in the measured data in the memory buffer.

8. The system according to claim 7, wherein memory buffer is piecewise transposed to eliminate gaps in the measured data in the memory buffer.

9. The system according to any of claims 1-8, wherein the voxels of the super voxel are chosen to substantially match the locations corresponding to memory cache lines which are used to update the super voxel.

10. The system according to any one of claims 1-9, the operations further including updating the plurality of voxels of the super voxel in parallel.

11. The system according to claim 1-9, the operations further including updating a plurality of super voxels in parallel.

12. The system according to any one of claims 1-11, wherein memory allocated to store the initial image is initialized as a constant value.

13. The system according to any one of claims 1-11, wherein memory allocated to store the initial image is initialized using a non-iterative reconstruction algorithm.

14. The system according to claim 13, wherein memory allocated to store the initial image is initialized using filtered-back projection (FPB),

15. The system according to any one of claims 1-11, wherein memory allocated to store the initial image is initialized using an iterative reconstruction algorithm.

16. The system according to any of claims 1-15, further comprising an irradiation source and a radiation sensor array, wherein the tomography system is configured to irradiate a test object using the irradiation source, measure resulting radiation using the radiation sensor array, and provide the measured data corresponding to the resulting radiation.

17. The system according to claim 16, wherein the tomography system is configured to rotate the irradiation source and the radiation sensor array around the test object to obtain the measured data.

18. The system according to any of claims 1-17, wherein the first memory comprises one or more of a computer cache memory located on the processor, a high speed ram buffer, and a GPU RAM memory.

19. The system according to claim 11, wherein the system utilizes augmented super voxel buffers to process the measured data.

20. The system according to claim 1, wherein super-voxels are updated in non-sequential order so as to speed convergence.

21. The system according to claim 20, wherein the super-voxels are updated based on the amount of change that occurred in the previous update of the super-voxel.

22. The system according to any of claims 1-21, wherein the voxels in the super voxel are substantially adjacent in the image.

23. A method for iterative reconstruction of computer tomography data, comprising: receiving computer tomography measured data, creating an initial image from the measured data using a computer processor, and updating spatially localized super-voxels in 2 or more dimensions, each super-voxel corresponding to a plurality of voxels whose corresponding measured data in memory substantially overlap.

24. The method according to claim 23, wherein said measured data forms a sinogram in 2 or more dimensions.

25. The method according to claim 23 or 24, wherein updates of super-voxels are performed non-sequentially.

26. The method according to any of claims 23-25, wherein the non-sequential updates of super-voxels depend on the amount of change that occurred in the previous update of the super-voxel.

27. The method according to any of claims 23-26, wherein intra-SV parallelism is used to process the data.

28. The method according to any of claims 23-27, wherein inter-SV parallelism is used to process the data.

29. The method according to claim 28, wherein augmented SVBs are utilized to process the data.

30. A method for iterative image reconstruction of computer tomography data, comprising: receiving computer tomography measured data and creating super-voxels with a size and shape so that memory access time is reduced, wherein the super-voxels corresponding measured data are localized in memory.

31. The method according to claim 30, wherein the super-voxels measured data are localized in memory so as to reduce time required to move said measurements to a super-voxel buffer and process the measurements in a super-voxel buffer.

32. The method according to claim 30 or 31, wherein the super-voxels size is selected to achieve the most rapid convergence of an iterative reconstruction algorithm for a predetermined processor configuration.

33. The method according to any of claims 30-32, wherein intra-SV parallelism is used to process the data.

34. The method according to any of claims 30-32, wherein inter-SV parallelism is used to process the data.

35. A method as in claim 34, wherein augmented SVBs are utilized to process the data.

36. A method for forward or back projection of computer tomography data, comprising: receiving computer tomography measured data and reorganizing stored measured data corresponding to a super-voxel so that the associated stored measured data are straightened to be in memory locations to allow efficient access by the computer, the super voxel comprising a plurality of image voxels whose corresponding stored measured data substantially overlap.

37. The method according to claim 36, wherein the stored measurements comprise a sinogram.

38. The method according to any one of claims 36-37, wherein the super-voxels pack efficiently into an image space in memory.

39. The method according to any one of claims 35-38, wherein the straightened memory locations are stored in one or more of a computer cache in a memory hierarchy, a high speed ram buffer, or a GPU RAM memory, that allows increased access speed to the measured data.

40. The method according to any one of claims 36-39, wherein the straightened memory forms a super-voxel buffer with high-speed access.

41. The method according to any one of claims 36-40, wherein piece-wise transposition of a super-voxel buffer is utilized to process the measured data.

42. The method according to any one of claims 36-41, wherein intra-SV parallelism is utilized to process the data.

43. The method according to any one of claims 36-41, wherein inter-SV parallelism is utilized to process the data.

44. The method according to claim 43, wherein augmented SVBs are utilized to process the data.
